SEQUENCE LISTING (51 sequences)

SEQ ID NO: 1 - protein, 5 aa, Mus musculus
DYGVS

SEQ ID NO: 2 - protein, 16 aa, Mus musculus
VIWGGGSTYYNSVLKS

SEQ ID NO: 3 - protein, 9 aa, Mus musculus
HEGSDAMDY

SEQ ID NO: 4 - protein, 5 aa, Mus musculus
DYVIS

SEQ ID NO: 5 - protein, 17 aa, Mus musculus
EIYPGSGSTYYNEKFKG

SEQ ID NO: 6 - protein, 10 aa, Mus musculus
SRGDYYAMDY

SEQ ID NO: 7 - protein, 5 aa, Mus musculus
SFGMH

SEQ ID NO: 8 - protein, 17 aa, Mus musculus
YISSDSSTIYYADTVKG

SEQ ID NO: 9 - protein, 12 aa, Mus musculus
EGGYDDYYAMDY

SEQ ID NO: 10 - protein, 5 aa, Mus musculus
DYYIN

SEQ ID NO: 11 - protein, 17 aa, Mus musculus
EIYPGSGDTYYNEKFKG

SEQ ID NO: 12 - protein, 14 aa, Mus musculus
YGYKGVYFDYWGQS

SEQ ID NO: 13 - protein, 11 aa, Mus musculus
KASQNVGTNVA

SEQ ID NO: 14 - protein, 7 aa, Mus musculus
RHPTGTV

SEQ ID NO: 15 - protein, 19 aa, Mus musculus
SNITAIVDVRWRHQAGNQT

SEQ ID NO: 16 - protein, 11 aa, Mus musculus
KASQDINNYIT

SEQ ID NO: 17 - protein, 7 aa, Mus musculus
YTSTLQP

SEQ ID NO: 18 - protein, 18 aa, Mus musculus
LQYDNLLTFGGGTKLEIK

SEQ ID NO: 19 - protein, 11 aa, Mus musculus
LASQTIGTWLA

SEQ ID NO: 20 - protein, 7 aa, Mus musculus
AATSLAD

SEQ ID NO: 21 - protein, 19 aa, Mus musculus
QQLYSIPLTFGGGTKLEIK

SEQ ID NO: 22 - protein, 12 aa, Mus musculus
SASSSVSSSYLH

SEQ ID NO: 23 - protein, 7 aa, Mus musculus
RTSNLAS

SEQ ID NO: 24 - protein, 19 aa, Mus musculus
QQWSGYPFTFGAGTKLELK

SEQ ID NO: 25 - protein, 15 aa, Unknown
  sequence from phage library
TGSDDLCYTFPCMHY

SEQ ID NO: 26 - protein, 17 aa, Unknown
  sequence from phage library
EAGKETCGYEWQYCPRP

SEQ ID NO: 27 - protein, 18 aa, Unknown
  sequence from phage library
EAGAYWCKAWGLSPCLVT

SEQ ID NO: 28 - protein, 19 aa, Unknown
  sequence from phage library
TGSGRMCDGSGPPTNCWFT

SEQ ID NO: 29 - protein, 20 aa, Unknown
  sequence from phage library
PGSQGYCHLNMRYQWICDSE

SEQ ID NO: 30 - DNA, 27 bp, Artificial Sequence
  primer
ggaggaagcggccgcggtactggcagc

SEQ ID NO: 31 - DNA, 26 bp, Artificial Sequence
  primer
cctcctctctagagcggaccaggagc

SEQ ID NO: 32 - DNA, 27 bp, Artificial Sequence
  primer
ggaggaagcggccgcacagargcwggt

SEQ ID NO: 33 - DNA, 26 bp, Artificial Sequence
  primer
cctcctctctagagnggytcmgtncc

SEQ ID NO: 34 - DNA, 27 bp, Artificial Sequence
  primer
ggaggaagcggccgcacagargcwggt

SEQ ID NO: 35 - DNA, 26 bp, Artificial Sequence
  primer
cctcctctctagagnggytcmgtncc

SEQ ID NO: 36 - DNA, 27 bp, Artificial Sequence
  primer
ggaggaagcggccgcacagargcwggt

SEQ ID NO: 37 - DNA, 26 bp, Artificial Sequence
  primer
cctcctctctagagnggytcmgtncc

SEQ ID NO: 38 - DNA, 27 bp, Artificial Sequence
  primer
ggaggaagcggccgcggtactggcagt

SEQ ID NO: 39 - DNA, 26 bp, Artificial Sequence
  primer
cctcctctctagagaggacccggggc

SEQ ID NO: 40 - DNA, 27 bp, Artificial Sequence
  primer
ggaggaagcggccgccgtcctggtagc

SEQ ID NO: 41 - DNA, 26 bp, Artificial Sequence
  primer
cctcctctctagagaggacgaggagc

SEQ ID NO: 42 - DNA, 27 bp, Artificial Sequence
  primer
ggaggaagcggccgcggcaccggtgac

SEQ ID NO: 43 - DNA, 26 bp, Artificial Sequence
  primer
cctcctctctagagagggccaggatc

SEQ ID NO: 44 - protein, 112 aa, Mus musculus
ESGPGLVAPSQSLSITCTVSGFSLTDYGVSWIRQPPGKGLEWLGVIWGGGSTYYNSVLKSRLSISKDNSKSQVFLKMNSLQTDDTAMYYCAKHEGSDAMDYWGQGTSVTVSS

SEQ ID NO: 45 - protein, 106 aa, Mus musculus
DIVMTQSQKFMSTSVGDRVSVTCKASQNVGTNVAWYQQKPGQSPKALISRHPTGTVESLIASQAVDLGQISLSPSAMCSVKTWQSISVSNITAIVDVRWRHQAGNQ

SEQ ID NO: 46 - protein, 119 aa, Mus musculus
QVQLQQSGPELVKPGASVKMSCKASGYTFTDYVISWVKQRTGQGLEWIGEIYPGSGSTYYNEKFKGKASLTADKSSNTAYMQLSSLTSADSAVYFCVRSRGDYYAMDYWGQGTSVTVSS

SEQ ID NO: 47 - protein, 106 aa, Mus musculus
DIQMTQSPSSLSASLGGKVTITCKASQDINNYITWYQHKPGKGPRLLIHYTSTLQPGIPSRFSGSGSGRDYSFSISNLEPEDIATYYCLQYDNLLTFGGGTKLEIK

SEQ ID NO: 48 - protein, 121 aa, Mus musculus
DVQLVESGGGLVQPGGSRKLSCAASGFTFSSFGMHWVRQAPEKGLEWVAYISSDSSTIYYADTVKGRFTISRDNPKNTLFLQMTSLRSEDTAMYYCAREGGYDDYYAMDYWGQGTSVTVSS

SEQ ID NO: 49 - protein, 107 aa, Mus musculus
DIQMTQSPASQSASLGESVTITCLASQTIGTWLAWYQQKPGKSPQLLIYAATSLADGVPSRFSGSGSGTKFSFKISSLQAEDFVSYYCQQLYSIPLTFGGGTKLEIK

SEQ ID NO: 50 - protein, 119 aa, Mus musculus
QVQLQQSGAELARPGASVKLSCKASGYTFTDYYINWAKQRTGQGLEWIGEIYPGSGDTYYNEKFKGKATLTADKSSSTAYMQLSSLTSEDSAVYFCARYGYKGVYFDYWGQSTTLTVSS

SEQ ID NO: 51 - protein, 108 aa, Mus musculus
ENVLTQSPAIMAASLGQKVTMTCSASSSVSSSYLHWYQQKSGASPKPLIHRTSNLASGVPARFSGSGSGTSYSLTISSVEAEDDATYYCQQWSGYPFTFGAGTKLELK

(19) United States
(12) Patent Application Publication
(10) Pub. No.: US 2011/0076723 A1
(43) Pub. Date: Mar. 31, 2011

(54) ANTIBODY-PEPTIDE FUSED SYNERGIBODY

(75) Inventors: Brian Hosung Min, Seongnam-si (KR); Jong Sang Ryu, Suwon-si (KR)

(73) Assignee: SAMSUNG ELECTRONICS CO., LTD., Suwon-si (KR)

(21) Appl. No.: 12/993,199

(22) PCT Filed: May 22, 2009
(86) PCT No.: PCT/KR09/02725

§ 371 Date: Nov. 17, 2010

(30) Foreign Application Priority Data
May 23, 2008 (KR) 10-2008-0048293

(51) Int. Cl. C12P 21/02; C07K 19/00; C07K 7/06; C07K 7/08; C07K 16/22; C12N 15/63; C12N 5/10; C07H 21/00

(52) U.S. Cl. 435/69.6; 530/391.7; 530/330; 530/326; 530/328; 530/327; 530/329; 530/389.2; 435/320.1; 435/325; 536/23.53; 536/23.1

(57) ABSTRACT

Disclosed herein are synergibodies, antibody-peptide fused protein molecules containing antibodies and bioactive peptides, which function in the occurrence or progress of the identical disease.

### TECHNICAL FIELD

This application relates to a fusion protein. More specifically, this application relates to a fusion protein wherein an antibody and a peptide are combined to produce a synergy effect.

### BACKGROUND ART

Recently, antibodies have been widely used for therapeutic purposes. Antibodies show excellent in vitro and in vivo stability characteristics and half-life profiles and can be expressed and produced in mass scale. Furthermore, antibody molecules are of dimeric structure and thus have very high avidity to antigens.

Although peptide based therapeutics can also be becoming increasingly popular due to their specificity and therapeutic potential, peptides by themselves as therapeutics present obstacles due to the unfavorable stability and half-life profiles. Therefore, additional means should be implemented to enhance the stability and the half-life profiles in order to use peptides as therapeutics.

Angiogenesis refers to the physiological process involving growth of new blood vessels from existing vessels. Angiogenesis is a normal process in growth and development, including wound healing. It is also a fundamental step in the transition of tumors from a dormant state to a malignant state.

### DISCLOSURE

### Technical Problem

Disclosed herein is to provide a fusion protein.

Disclosed herein is also to provide a synergibody wherein an antibody and a peptide are combined.

### Technical Solution

An embodiment of the fusion protein disclosed herein is an antibody-peptide fused synergibody including an antibody and a biologically active peptide, which functions in the occurrence or progress of the same disease.

An embodiment of the pharmaceutical composition disclosed herein is a pharmaceutical composition containing an antibody-peptide fusion protein as an effective ingredient, wherein the antibody-peptide fusion protein includes an antibody and a biologically active peptide, wherein the antibody and the biologically active peptide function during the occurrence or progress of the same disease.

An embodiment of the method of producing an antibody-peptide fused synergibody disclosed herein is a method of producing antibody-peptide fused synergibodies, including inserting nucleic acid molecules encoding the antibody-peptide fused protein into a vector; introducing the vector into a host cell; and culturing the host cells.

### Advantageous Effects

The Antibody-peptide synergibody disclosed herein has a synergy effect by functioning in occurrence or progress of the same disease.

### Best Mode

The term “fused synergibody” as used in this specification refers to a fusion protein wherein two or more proteins are combined and, as a result of the combination, the function of each of the proteins improves. The term “improve” means “ameliorate,” “increase,” relative to separate proteins or “get superior,” or “get stronger,” than separate proteins, etc. The “improve” may be any improvement regardless of the extent of the improvements.

The “antibody” as used in this specification refers to any substance produced by the stimulation by an antigen in an immune system and is not limited to any specific species. The “antibody” includes animal antibodies, chimeric antibodies, humanized antibodies or fully human antibodies. Also, the antibody includes any fragment of the antibodies retaining the antigen-binding affinity.

The “peptide” as used in this specification refers to any amino acid polymer wherein amino acids are connected by peptide bonds. The length of the peptide is not limited to any specific range.

The term “Complementarity-determining regions (CDR)” refers to a region which offers a binding specificity to an antigen among variant regions of antibodies.

The term “target” as used in this specification refers to the subject which the antibody or peptide is combined to and thereby effects on, i.e., biomolecules such as DNA, RNA, protein, etc. “Target” may also refer to the ultimate purpose of injecting the antibody or peptide, i.e., a particular disease, its treatment or prevention. For example, the target may be angiogenesis or cancer.

The term “bioactive” or “biologically active” as used in this specification means showing biological activity in the body to carry out a specific function. For example, it may mean the combination with a particular biomolecule such as protein, DNA, etc., and then promotion or inhibition of the activity of such biomolecule.

The term “connected” includes covalently bonded as well as non-covalently bonded. Also, “connected” includes directly connected as well as mediated by a mediator such as a linker.

Angiogenesis in a cancer tissue involves the steps of cooption, wherein the oncogene selects an existing blood vessel to form new blood vessels in a cancer tissue, and vessel regression, wherein the coopted existing vessel is destroyed through angiopoietin-2 (Ang-2) pathway. The blood vessel regression causes hypoxia in the environment within the cancer tissue, which is an environment wherein the formation of angiogenesis is possible. Under such condition, the expression of the vascular endothelial cell growth factor (VEGF) is increased, and new blood vessels are formed.

Angiopoietin is a growth factor that promotes the formation of new vessels. Ang-1, Ang-2, Ang-3 and Ang-4 are well-known angiopoietins. Ang-1 and Ang-2 are required for the formation of mature blood vessels. Ang-2 is also known as ANGPT2, and both ANGPT1 and TIE2 are naturally occurring antagonists that are expressed only in the blood vessel remodeling areas.

Vascular endothelial cell growth factor (VEGF) is a type of platelet-derived growth factor and is a material signal protein related to the formation of angiogenesis.

The essence lies in the combination of only the positive characteristics of antibodies and peptides. Antibodies have been widely used as therapeutics recently. Antibodies show excellent in vitro and in vivo stability characteristics and half-life profiles and can be expressed and produced in mass scale. Furthermore, antibody molecules are of dimeric nature and thus have very high avidity to antigens.

Meanwhile, therapeutics based on peptides having antigen binding capacity are becoming increasingly popular due to their specificity and therapeutic potential. However, peptides by themselves as therapeutics present obstacles due to the unfavorable stability and half-life profiles. A synergibody according to an embodiment disclosed herein combines only the positive characteristics of antibodies and the positive characteristics of peptides.

A synergibody according to an embodiment disclosed herein may include a bioactive peptide of 7 to 50 amino acids. This is because if more than 50 amino acids are combined, the fused antibody's functionality may be undermined, and if less than 7 amino acids are combined, its antigen affinity is clearly decreased. The peptides may be combined with antibodies by DNA recombinant fusion. Peptides may be linked to antibody heavy chains or light chains, preferably to the C-terminus of heavy chains.

In a synergibody according to an embodiment disclosed herein, the peptide and the antibody may have capacities to bind different targets. In other words, the antibody moiety of a synergibody may bind one target while the peptide moiety binds another target. Then, the synergibody will be able to bind two target molecules simultaneously in one place. The antibody moiety and peptide moiety of the synergybody may show similar biological effects in the occurrence or progress of the same disease and create a synergy effect. Meanwhile, due to the dimeric structure of the antibody moiety of the synergibody, a companion dimeric structure of the peptide moiety can also be induced, thereby causing a synergistic effect—very high avidity of the entire fused protein to each target.

The synergibody according to an embodiment disclosed herein includes antibodies and peptides linked to each other directly as well as through a linker. It also includes the gene encoding an antibody and the gene encoding a peptide linked by a linker DNA sequence.

In an embodiment, the antibody is an antibody having a binding affinity to Ang-2, an antibody inhibiting Ang-2 or an antibody binding to Ang-2. The peptide is a bioactive peptide that has a binding affinity to VEGFR-2, inhibits the binding between VEGF and VEGFR-2, binds to VEGFR-2 or blocks the activation of VEGFR-2.

The complementarity determining regions (CDRs) of the heavy chins of the antibody having a binding affinity to Ang-2, the antibody inhibiting Ang-2 or the antibody binding to Ang-2 disclosed herein have, for example, the following amino acids:

TABLE 1

| | Amino acid sequence of heavy chain | | |
|---|---|---|---|
| Antibody name | CDR1 | CDR2 | CDR3 |
| SAITAng-2-1 | DYGVS | VIWGGGSTYYNSVLKS | HEGSDAMDY |
| | (SEQ ID NO: 1) | (SEQ ID NO: 2) | (SEQ ID NO: 3) |
| SAITAng-2-2 | DYVIS | EIYPGSGSTYYNEKFKG | SRGDYYAMDY |
| | (SEQ ID NO: 4) | (SEQ ID NO: 5) | (SEQ ID NO: 6) |
| SAITAng-2-3 | SFGMH | YISSDSSTIYYADTVKG | EGGYDDYYAMDY |
| | (SEQ ID NO: 7) | (SEQ ID NO: 8) | (SEQ ID NO: 9) |
| SAITAng-2-4 | DYYIN | EIYPGSGDTYYNEKFKG | YGYKGVYFDYWG |
| | (SEQ ID NO: 10) | (SEQ ID NO: 11) | QS |
| | | | (SEQ ID NO: 12) |

The CDRs of the heavy chins of the antibody having a binding affinity to Ang-2, the antibody inhibiting Ang-2 or the antibody binding to Ang-2 disclosed herein have, for example, the following amino acids:

TABLE 2

| | Amino acid sequence of light chain | | |
|---|---|---|---|
| Antibody name | CDR1 | CDR2 | CDR3 |
| SAITAng-2-1 | KASQNVGTNVA | RHPTGTV | SNITAIVDVRWRHQAGNQ |
| | (SEQ ID NO: 13) | (SEQ ID NO: 14) | T |
| | | | (SEQ ID NO: 15) |
| SAITAng-2-2 | KASQDINNYIT | YTSTLQP | LQYDNLLTFGGGTKLEIK |
| | (SEQ ID NO: 16) | (SEQ ID NO: 17) | (SEQ ID NO: 18) |
| SAITAng-2-3 | LASQTIGTWLA | AATSLAD | QQLYSIPLTFGGGTKLEIK |
| | (SEQ ID NO: 19) | (SEQ ID NO: 20) | (SEQ ID NO: 21) |
| SAITAng-2-4 | SASSSVSSSYLH | RTSNLAS | QQWSGYPFTFGAGTKLEL |
| | (SEQ ID NO: 22) | (SEQ ID NO: 23) | K |
| | | | (SEQ ID NO: 24) |

Also, the bioactive peptides that has a binding affinity to VEGFR-2, inhibits the binding between VEGF and VEGFR-2, binds to VEGFR-2 or blocks the activation of VEGFR-2 have, for example, the following amino acids:

TABLE 3

| Peptide name | Amino acid sequence | |
|---|---|---|
| DTN6VEGFR-2-1 | TGSDDLCYTFPCMHY | (SEQ ID NO: 25) |
| DTN8VEGFR-2-2 | EAGKETCGYEWQYCPRP | (SEQ ID NO: 26) |
| DTN9VEGFR-2-3 | EAGAYWCKAWGLSPCLVT | (SEQ ID NO: 27) |
| DTN10VEGFR-2-4 | TGSGRMCDGSGPPTNCWFT | (SEQ ID NO: 28) |
| DTN11VEGFR-2-5 | PGSQGYCHLNMRYQWICDSE | (SEQ ID NO: 29) |

In another embodiment, the antibody may be a monoclonal antibody. Also, the antibody may be a chimeric antibody, humanized antibody or a fully human antibody. Further, the antibody includes a fragment of the antibody as mentioned above.

Amino acid sequence modification(s) of the antibodies or the bioactive peptides described herein are contemplated. For example, it may be desirable to improve the binding affinity and/or other biological properties of the antibody and the bioactive peptide.

Amino acid sequence variants of the antibody or the bioactive peptide are prepared by introducing appropriate nucleotide changes into the antibody nucleic acid or the bioactive peptide nucleic acid, or by peptide synthesis. Such modifications include, for example, deletions from, and/or insertions into and/or substitutions of, residues within the amino acid sequences of the antibody or the bioactive-peptide. Any combination of deletion, insertion, and substitution is made to arrive at the final construct, provided that the final construct possesses the desired characteristics. The amino acid changes also may alter post-translational processes of the antibody or the bioactive peptide, such as changing the number or position of glycosylation sites.

A useful method for identification of certain residues or regions of the antibody or the bioactive peptide that are preferred locations for mutagenesis is called “alanine scanning mutagenesis” as described by Cunningham and Wells Science, 244: 1081-1085 (1989). Here, a residue or group of target residues are identified (e. g., charged residues such as arg, asp, his, lys, and glu) and replaced by a neutral or negatively charged amino acid (most preferably alanine or polyalanine) to affect the interaction of the amino acids with Ang-2 antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-Ang2 antibody or the bioactive peptide variants are screened for the desired activity.

Another type of variant is an amino acid substitution variant. These variants have at least one amino acid residue in the anti-Ang-2-antibody or the bioactive peptide molecule replaced by a different residue. The sites of greatest interest for substitutional mutagenesis include the hypervariable regions, but FR alterations are also contemplated.

Conservative substitutions are shown in Table 4 under the heading of “preferred substitutions”. If such substitutions result in a change in biological activity, then more substantial changes, denominated “exemplary substitutions” in Table 4, or as further described below in reference to amino acid classes, may be introduced and the products screened.

TABLE 4

| | | Preferred Residue |
|---|---|---|
| Original | Exemplary Residue Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody or the bioactive peptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and (6) aromatic: trp, tyr, phe.

Non-conservative substitutions will entail exchanging a member of one of these classes for another class.

Any cysteine residue not involved in maintaining the proper conformation of the anti-Ang-2 antibody or the bioactive peptide also may be substituted, generally with serine, to improve the oxidative stability of the molecule and prevent aberrant crosslinking. Conversely, cysteine bond (s) may be added to the antibody or the bioactive peptide to improve its stability (particularly where the antibody is an antibody fragment such as an Fv fragment).

Another type of amino acid variant of the antibody or the bioactive peptide alters the original glycosylation pattern of the antibody or the bioactive peptide.

By altering is meant deleting one or more carbohydrate moieties found in the antibody or the bioactive peptide, and/or adding one or more glycosylation sites that are not present in the antibody or the bioactive peptide.

Glycosylation of antibodies or bioactive peptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody or the bioactive peptide is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody or the bioactive peptide (for O-linked glycosylation sites).

An isolated nucleic acid encoding the humanized anti-Ang-2 antibody, vectors and host cells comprising the nucleic acid, and recombinant techniques for the production of the antibody are also provided.

For recombinant production of the antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

Suitable host cells for cloning or expressing the DNA in the vectors herein are the prokaryote, yeast, or higher eukaryote cells.

Host cells are transformed with the above-described expression or cloning vectors for anti-Ang-2 antibody production and cultured in conventional nutrient media modified as appropriate for inducing promoters, selecting transformants, or amplifying the genes encoding the desired sequences.

The method of producing synergibodies disclosed herein is described below.

### 1. Preparation and Screening of Antibodies

The antibodies may be monoclonal antibodies. Antibodies may be prepared through methods well-known in the relevant field of technology. For instance, Anti-Ang-2 antibodies may be generated using mouse monoclonal antibodies based on a procedure described by Schwaber et. al. (Schwaber, J and Cohen, E. P., “Human×Mouse Somatic Cell Hybrid Clones Secreting Immunoglobulins of Both Parental Types,” Nature, 244 (1973), 444-447.).

Individual monoclonal antibodies may be screened using a typical Enzyme-Linked ImmunoSorbent Assay (ELISA) format based on their Ang-2 binding capacity. The binders may be screened for inhibition activity using a functional assay such as a competitive ELISA for examining molecular interaction. Then, these monoclonal antibody members selected based on strong inhibition activity may be tested for their respective affinities to Ang-2 (Kd values).

The final selected clones may become chimeric antibodies having human immunoglobulin G₁ (IgG₁) substituted for portions except for antigen-binding sites. Antibody chimerization processes are well known in the art. For example, antibody chimerization may be accomplished by the method disclosed in published patents such as EP0355068, EO0363712 and EP0491351, the disclosure of each is incorporated herein by reference in its entirety. Chimeric antibodies may be tested for inhibition activity through functional assay such as a cell-based assay. In addition to chimerized antibodies, humanized antibodies may also be produced and used. The production process of humanized antibodies is well known in the art, for example, by Almagro et. al. (Almagro, J. C. and Fransson, J., “Humanization of antibodies,” Frontiers in Bioscience, 13 (2008), 1619-1633.), the disclosure of which is incorporated herein by reference in its entirety.

### 2. Screening of Peptides

Peptides may be screened, for instance, from known phage display library. For example, phage library may be added to beads coated with VEGFR-2, phage that binds with VEGFR-2 may be selected therefrom and amino acid sequences may be obtained from such selected phages by cloning DNA sequences encoding the peptides.

Meanwhile, individual peptides may be screened by using a typical ELISA format, based on the VEGFR-2-binding capacity. The binders may be screened for inhibition activity using a functional assay such as competitive ELISA for molecular interaction or cell-based functionality assay.

### 3. Preparation of Fused Protein Synergibodies

Genes that link DNA sequences encoding selected antibodies and DNA sequences encoding peptides may be obtained using recombinant DNA technology. A recombinant DNA sequence so obtained may be inserted into a vector. Fused protein may be prepared by introducing such vector into host cells and culturing such cells.

Individual fused protein synergibodies may be screened for capacity to simultaneously bind two targets using a typical ELISA format based on their Ang-2 binding capacity and VEGFR-2 binding capacity. The binders may be screened for inhibiting activity using a functional assay such as molecular interaction or cell-based assay. Then, these selected members based on strong inhibition activity may be tested for synergistic effect.

Pharmaceutical composition of the fusion protein may be prepared for storage by mixing the fusion protein having the desired degree of purity with optional physiologically acceptable carriers, excipients or stabilizers {Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA.

The composition herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the fusion polypeptide, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical composition is administered to a mammal, preferably a human being, by any suitable means well known in the art, including intravenous, intramuscular, intraperitoneal, intracerebrospinal, subcutaneous, intraarticular, intrasynovial, intrathecal, oral, tropical, or intranasal administration. Preferably the dosing is given by injections, most preferably intravenous or subcutaneous injections, depending in part on whether the administration is brief or chronic.

For the prevention or treatment of disease, the appropriate dosage of the fusion protein will depend on the type of disease to be treated, the severity and course of the disease, whether the fusion polypeptide is administered for preventive or therapeutic purposes, previous therapy, the patient's clinical history and response to the fusion polypeptide, and the discretion of the attending physician. The fusion polypeptide is suitably administered to the patient at one time, or over a series of treatments.

Depending on the type and severity of the disease, about 1 μg/kg to 20 mg/kg (e.g., 0.1-15 mg/kg) of fusion polypeptide is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Mode for Invention

### EXAMPLES

### Example 1

### Generation of Ang-2 Inhibiting Antibody and Test for Inhibiting Activity

### 1. Preparation of Anti-Ang-2 Mouse Antibodies

Ang-2 proteins secreted from human umbilical vein endothelial cells (HUVECs) are administered to a BALB/c mouse (5 week aged, female) to artificially induce immune response and then hybridomas producing individual antibodies are based on the procedure described by Schwaber et. al. (Schwaber, J and Cohen, E. P., “Human X Mouse Somatic Cell Hybrid Clones Secreting Immunoglobulins of Both Parental Types,” Nature, 244 (1973), 444-447.), disclosure of which is incorporated herein by reference in its entirety.

### 2. Selection of Hybridomas Generating Anti-Ang-2 Antibodies and Antibody Purification

The individual antibody generating hybridomas are screened and 18 hybridomas generating anti-Ang-2 monoclonal antibodies (3A5F, 6G2F, 15G1F, 17B1F, 21A3F, 25C2F, 28H5F, 30H3F, 1B2F, 8E4F, 16B1F, 22E9F, 23A4F, 24D5F, 26E4F, 27H1F, 29E1F, 31F4F) are selected from among the 1,824 hybridomas differentiated from 19 parent hybridomas, using a typical ELISA format based on the Ang-2 binding capacity. Each hybridoma is cultivated in DMEM (Dulbeco's Modified Eagle's Medium), the culture solution is collected, and anti-Ang-2 monoclonal antibodies are purified by Protein G-affinity chromatography.

### 3. Ang-2: Tie-2 Neutralization ELISA (Competitive ELISA)

A competitive ELISA is performed to assess the molecular interaction of the binders. A 96-well MaxiSorp™ flat-bottom plate (Nunc) is coated with hTie2-Fc (R&D Systems), which is a fused protein including the water-soluble extracellular portion of human Tie-2 and Fc of human IgG₁ dissolved in DMEM culture solution containing 50 μg/ml bovine serum albumin (BSA). The concentration of hTie2-Fc used as a coating material is equivalent to 75% of its maximum binding concentration with 1 nM of recombinant hAng-2 (recombinant protein with His tag attached to human Ang-2; R&D Systems). Then the plate is washed five times with Phosphate Buffer Saline (PBS) containing 0.1% Tween-20 and blocked at room temperature for two hours with PBS containing 5% BSA. Each anti-Ang-2 antibodies are diluted from 100 nM by one-fourth each time with PBS containing 1% BSA and 1 nM hAng-2 and adjusted to 6.1 pM for Ang-2:Tie-2 neutralization ELISA. 100 μl of antibody/hAng-2 solutions are placed in each corresponding well and left to react at room temperature for two hours. Then the wells are washed five times with PBS containing 0.1% Tween-20, and the anti-His antibodies (GE Healthcare) are diluted to about 1:3,000 with PBS containing 1% BSA and left to react at room temperature for one hour. Afterwards, 100 μl of goat anti-mouse-IgG-HRP (Pierce) diluted with PBS containing 1% BSA to about 1:10,000 is added to each well of the plate, left to react at room temperature for one hour, and washed with PBS containing 0.1% Tween-20 five times. Lastly, 100 μl of TMB substrate (SIGMA) is added to each well of the plate to induce color reaction, and the reaction is stopped by adding 50 μl of 5N H₂SO₄ solution. The OD₄₅₀ value is read on a plate reader (Molecular Devices). Through the foregoing, the 50% inhibition concentration, IC₅₀, is obtained for the Ang-2:Tie-2 binding capacity. The results are as shown in Table 5 below.

TABLE 5

| | 50% inhibition concentration |
|---|---|
| | (IC₅₀, nM) for Ang-2:Tie-2 |
| Name of Antibody | binding capacity |
| Unrelated control group | >100 |
| SAITAng-2-1 | 0.764 |
| SAITAng-2-2 | 1.859 |
| SAITAng-2-3 | 0.822 |
| SAITAng-2-4 | 1.012 |

4. Measuring the Affinity to Antigens (Kd Values) using a Surface Plasmon Resonance (SPR) Method

The SPR method using BIAcore T100 (GE Healthcare) is used to accurately measure the affinity to the Ang-2 antigens. 20 μg/ml recombinant hAng-2 (R&D Systems) is fixed on a sensor chip CM5 (GE Healthcare) using pH4.5 acetate solution and amine coupling kit (GE Healthcare). The antibodies obtained in Section 2 above are diluted to eight different concentrations down to 0.78125 nM by diluting to ½ each time starting from 100 nM. Then the antigen/antibody affinity is measured by causing the antigens to repeatedly bind and disassociate with the antibodies fixed on the sensor chip (using pH2.2 glycine-HCl solution). The results are as shown in Table 6 below.

TABLE 6

| | | | Affinity |
|---|---|---|---|
| Name of Antibody | On rate (1/Ms) | Off Rate (1/s) | (Kd, nM) |
| SAITAng-2-1 | 6.135 × 10⁵ | 2.901 × 10⁻⁴ | 0.473 |
| SAITAng-2-2 | 4.332 × 10⁵ | 4.769 × 10⁻⁴ | 1.101 |
| SAITAng-2-3 | 5.012 × 10⁵ | 3.023 × 10⁻⁴ | 0.603 |
| SAITAng-2-4 | 5.121 × 10⁵ | 4.769 × 10⁻⁴ | 0.931 |

### 5. Cloning Anti-Ang-2 Mouse Antibody Genes

The entire RNA is obtained from each antibody generating hybridoma (2×10⁶ cells) obtained in Section 2 above, using RiboPure™ Kit (Ambion, cat#AM1924). Then using them as a template, primary single-strand cDNA is synthesized using Protoscript® First Strand cDNA Synthesis Kit (New England Biolab). Afterwards, the following program is run by using a thermocycler (GeneAmp PCR System 9700, Applied Biosystem) and mouse IgPrimer Set (Novagen), and only the gene sequences of the variable region of the heavy chains and light chains of the monoclonal antibodies generated from each hybridoma are amplified: 94° C. for 5 minutes; [94° C. for 1 minute, 60° C. for 1 minute, 72° C. for 2 minutes]×35 cycles; at 72° C. for 6 minutes; and cooled to 4° C. The PCR products obtained from each reaction are cleaned up using QIAquick Multiwell PCR Purification kit (Qiagen) pursuant to the manufacturer's protocol.

The PCR products obtained in accordance with the above are cloned into pGEM-T Easy (Promega) and DNA sequencing is performed in accordance with the published method. As a result, the CDR sequences in Tables 1 and 2 are obtained.

### 6. Expression and Purification of Chimeric Antibodies

The variable regions of the heavy chains and light chains prepared in Section 5 above are cloned into different vectors. The variable region of the heavy chain includes a CMV promoter (cytomegalovirus promoter) and is cloned into a vector including Fc region and constant region of human IgG₁. The light chain includes a CMV promoter and is cloned into a vector including the constant region of human IgG1. Specifically, the heavy chain and the vector including the heavy chain are treated with restriction enzymes of SfiI (Roche) and NheI (Roche), the light chain and the vector including the light chain are treated with restriction enzymes of SfiI (Roche) and BglII (Roche), and ligated with T4 DNA Ligase (New England Biolab) to prepare heavy chain vectors and light chain vectors for expression of chimeric antibodies including the desired variable region.

The heavy chains and light chains obtained in accordance with the above are transfected into HEK-293E cells (Korea Research Institute of Bioscience and Biotechnology). The cells are cultivated in Dulbecco's Modified Essential Medium (DMEM, Invitrogen) that does not contain any serum, and the medium is removed and replenished four times at three-day intervals. The culture medium so obtained contains chimeric antibodies including heavy chains and light chains including the variable region sequences of SEQ ID NOs: 44, 46, 48 or 50 (heavy chain) and SEQ ID NOs: 45, 47, 49 or 51 (light chain), and CDRs of Tables 1 and 2. The culture medium containing expressed chimeric antibodies is centrifuged to remove the remaining cells and impurities, and purified through affinity chromatography using Protein A which has strong binding capacity with Fc-region of the antibodies.

### 7. Cell-Based Functional Assay

Chimeric antibodies may be screened by functional assays such as cell-based assay for inhibition activity. In this example, BD BioCoat™ Angiogenesis System-Endothelial Cell Tube Formation (BD Biosciences) is used. BD Falcon 96-well Black/Clear plate coated with BD Matrigel Matrix that had been stored at −20° C. is slowly melted at 4° C. for 6 hours, and the mat cover of the plate is removed inside a sterile clean bench. Then it is left at 37° C. , 5% CO₂ for 30 minutes so that the Matrigel™ Matrix would form polymers. Then it is made into a suspension by treating HUVEC, cultured on a petridish containing EGM-2 MV (Lonza) medium added with 10% fetal bovine serum to 70 to 80% confluence, with 0.5% Trypsin-EDTA (Gibco). Then it is resuspended in a culture medium containing 10% fetal bovine serum to a cell concentration of 4×10⁵ cells/ml. At this time, 5 μM of each anti-Ang-2 chimeric antibodies expressed and purified in Section 5 above are also added and resuspended. Then 50 μl (about 2×10⁴ cells) of each of the suspension is added to each well of the plate and incubated at 37° C. , 5% CO₂ for 18 hours. Then the medium is removed and each well is washed twice with 100 μl of Hanks' balanced salt solution (HBSS, Invitrogen) ensuring that the tubes of the cells formed on BD Matrigel™ Matrix are not damaged. 50 μl of calcein AM (BD sciences) dissolved in HBSS at the concentration of 8 μg/ml is added to each well. The plate is then left to react at 37° C., 5% CO₂ for 30 minutes and the calcein AM (BD sciences) solution is removed and washed twice with HBSS. Lastly, an image of the length of the entire tube of HUVEC formed on BD Matrigel™ Matrix is obtained on a fluorescent microscope and analyzed by using MetaMorph® software (Universal Imaging Corporation™, http://www.moleculardevices.com). The results are as shown in Table 7 below.

TABLE 7

| | Length of the entire tube of HUVEC |
|---|---|
| Name of Antibody | (2 × 10⁴ cells) (pixels) |
| Unrelated control group | 12,227 ± 1,232 |
| SAITAng-2-1 (chimeric) | 5,139 ± 832 |
| SAITAng-2-2 (chimeric) | 7,721 ± 773 |
| SAITAng-2-3 (chimeric) | 5,012 ± 984 |
| SAITAng-2-4 (chimeric) | 6,117 ± 1,043 |

### Example 2

### Screening of Bio-Active Peptides that Bind VEGFR-2 and Test for their Activity

VEGFR-2 binding peptides are selected from random peptide phage display library. Peptide phage display process is carried out as follows.

### 1. VEGFR-2-Coated Magnetic Bead Preparation

### 1A. VEGFR-2 Immobilization on Magnetic Beads

For non-specific elution (see below section 2D), the biotinylated recombinant VEGFR-2 protein (protein having the water soluble extracellular site of human VEGFR-2 and Fc of human IgG1 connected thereto, R&D Systems) is immobilized on the Streptavidin Dynabeads, Dynal). For first selection, total three rounds are conducted. The concentration of the biotinylated recombinant VEGFR-2 protein for the first round is 4 mg per 100 ml of the bead stock.

For antigen (VEGFR-2)-specific elutions (see below section 2E), 2 mg of biotinylated recombinant VEGFR-2 protein is immobilized on 50 ml of the Streptavidin Dynabeads for the second rounds of selection. The coating concentration is reduced to 1 mg of biotinylated VEGFR-2 protein per 50 ml of the bead stock for the third rounds of selection (see below section 2E). By drawing the beads to one side of a tube using a magnet and pipetting away the liquid, the beads is washed five times with the phosphate buffer saline (PBS) and resuspended in PBS.

The biotinylated VEGFR-2 protein is added to the pre-washed beads at the above concentration and incubated with rotation for 1 hour at room temperature followed by a few hours to an overnight incubation at 4° C. with rotation. VEGFR-2 coated beads are then blocked by adding BSA to 1% final concentration and incubate overnight at 4° C. with rotation. The resulting VEGFR-2 coated beads are then washed five times with PBS before being subjected to the selection procedures.

### 1B. Negative Selection Bead Preparation

Additional beads are prepared for negative selections. For each panning condition, 500 μl of the bead stock from the manufacturer is subjected to the above procedure (section 1A) except that the incubation step with biotinylated VEGFR-2 is omitted. In the last washing step, the beads are divided into five 100 μl aliquots.

### 2. Selection of VEFGR-2 Binding Phage

### 2A.

Seven (Table 8) peptide phage libraries (Peptide phage display library, Dyax Corp), are used to select for VEGFR-2 binding phage.

TABLE 8

| library | Diversity |
|---|---|
| TN6/VII | 1.2 × 10⁹ independent transformants |
| TN7/IV | 2.3 × 10⁹ independent transformants |
| TN8/IX | 5.0 × 10⁹ independent transformants |
| TN9/IV | 3.2 × 10⁹ independent transformants |
| TN10/X | 2.0 × 10⁹ independent transformants |
| TN11/I | 2.7 × 10⁹ independent transformants |
| TN12/I | 1.4 × 10⁹ independent transformants |

Each library is subjected to non-specific elution (see below section 2D) and antigen elution (VEGFR-2, see below section 2E). Therefore, six different panning conditions are carried out for the screening of phages binding VEFGR-2. For all three libraries, the phages from the first round of selection are eluted only in a non-specific manner for further rounds of selection in the first selection. The VEGFR-2 specific phage elution is used in the second and third rounds of selection (see below section 2E).

### 2B. Negative Selection

For each panning condition, about 100 random library equivalent for libraries (1.2×10¹¹ pfu for TN6/VII, 2.3×10¹¹ pfu for TN7/IV, 5×10¹¹ pfu for TN8/IX, 3.2×10¹¹ pfu for TN9/IX, 2.0×10¹¹ pfu for TN10/X, 2.7×10¹¹ pfu for TN11/I, and 1.4×10¹¹ pfu for TN12/I) are aliquoted from the library stock and diluted to 400 μl with PBS-T (PBS with 0.05% Tween-20). The 400 μl diluted library stock is added to the beads prepared for negative selections (section 1B). The resulting mixture is incubated for 10 minutes at room temperature with rotation. The phage supernatant is drawn out and collected while immobilizing the beads using the magnet and 100 μl of PBS-T is added to further collect the phage supernatant. In this way, five more negative selection steps are performed.

2C. Selection using the VEGFR-2 Protein Coated Beads

The phage supernatant after the last negative selection step (section 1B) is added to the VEGFR-2 coated beads after the last washing step (section 1A). This mixture is incubated with rotation for one to two hours at room temperature, allowing specific phage to bind to the target protein. After the supernatant is discarded, the beads are washed about ten times with PBST followed by twice with PBS-T.

### 2D. Non-Specific Elution

After the final washing liquid is drawn out (section 2C), 1 ml of Min A salts solution (60 mM, K₂HPO₄, 33 mM KH₂PO₄, 7.6 mM (NH₄)₂SO₄, and 1.7 mM sodium citrate) is added to the beads. Then, this bead mixture is added directly to a concentrated bacteria sample for infection (see below section 3A and 3B).

### 2E. Antigen (VEGFR-2) Elution of Bound Phage

For round 2, after the last washing step (section 2C), the bound phages are eluted from the magnetic beads by adding 100 ml of 1 pM, 0.1 nM, and 10 nM recombinant VEGFR-2 protein successively with a 30-minute incubation for each condition. The remaining phages are eluted nonspecifically (section 2D). The eluted phages from 10 nM and non-specific elutions are combined and subject to the third round of selection (see below section 4).

For round 3, after the last washing step (section 2C), the round phages are eluted from the magnetic beads by adding 100 μl of 2% BSA, 1 nM recombinant VEGFR-2 protein, and 10 nM recombinant VEGFR-2 protein successively with a 30-minute incubation for each condition. In addition, the phages are eluted with 1 ml of 100 mM triethylamine solution (Sigma) for 10 minutes on a rotator. The pH of the phage containing solution is neutralized with 0.5 ml of 1 M Tris-HCI (pH 7.5). After the last elution with 100 mM triethylamine solution, the remaining phages are eluted non-specifically (section 2D).

### 3. Amplification

### 3A. Preparation of Plating Cells

Fresh E. coli. (XL-1 Blue MRF') culture is grown to OD₆₀₀=0.5 in LB media containing 12.5 mg/ml tetracycline. For each panning condition, 20 ml of this culture is chilled on ice and centrifuged. The bacteria pellet is resuspended in 1 ml of the Min A Salts solution.

### 3B. Transduction

Each mixture from different elution methods (section 2D, 2E and 2F) is added to a concentrated bacteria sample (section 3A) and incubated at 37° C. for 15 minutes. 2 ml of NZCYM media (2×NZCYM, 50 μg/ml Ampicillin) is added to each mixture and incubate at 37° C. for 15 minutes. The resulting 4 ml solution is plated on a large NZCYM agar plate containing 50 μg/ml Ampicillin and incubated overnight at 37° C.

### 3C. Phage Harvesting

Each of the bacteria/phage mixture grown overnight on a large NZCYM agar plate (section 3B) is scraped off in 35 ml of LB media, and the agar plate is further rinsed with additional 35 ml of LB media. The resulting bacteria/phage mixture in LB media is centrifuged to pellet the bacteria away. 50 ml of the phage supernatant is transferred to a fresh tube, and 12.5 ml PEG solution (20% PEG8000, 3.5M ammonium acetate) is added. After incubating on ice for 2 hours, the precipitated phages is centrifuged down and resuspended in 6 ml of the phage resuspension buffer (250 mM NaCl, 100 mM Tris ph8, 1 mM EDTA). This phage solution is further purified by centrifuging away the remaining bacteria and precipitating the phage for the second time by adding 1.5 ml of the PEG solution. After a centrifugation step, the phage pellet is resuspended in 400 μl of PBS. This solution is subjected to a final centrifugation to rid of remaining bacteria debris. The resulting phage preparation is titered by a standard plaque formation assay (Molecular Cloning, Maniatis et al 3^{rd} Edition).

### 4. Two More Rounds of Selection and Amplification

In the second round, the amplified phage (10¹⁰ pfu) from the first round (section 3C) is used as the input phage to perform the selection and amplification steps (section 2 and 3). For the VEGFR-2 elution, phages from 10 nM and non-specific elutions are combined and amplified for the third round of selection. The amplified phage (10⁹ pfu) from the second round in turn is used as the input phage to perform third round of selection and amplification (sections 2 and 3). After the elution steps (sections 2D and 2E) of the third round, a small fraction of the eluted phage is plated out as in the plaque formation assay (section 3C). Individual plaques are picked and placed into 96 well microtiter plates containing 100 μl of TE buffer in each well. These master plates are incubated at 4° C. overnight to allow phage to elute into the TE buffer.

### 5. Clonal Analysis (Phage ELISA and DNA Sequencing)

The phage clones are analyzed by phage ELISA and sequencing methods. The sequences are ranked based on the combined results from these two assays. As a result, the amino acid sequences of Table 3 are obtained

### 5A. Phage ELISA

An XL-1 Blue MRF' culture is grown until OD₆₀₀ reached 0.5. 30 μl of this culture is aliquoted into each well of a 96 well microtiter plate. 10 μl of eluted phage (section 4) is added to each well and allowed to infect bacteria for 15 minutes at room temperature. About 100 μl of LB media containing 12.5 mg/ml of tetracycline and 50 mg/ml of ampicillin is added to each well. The microtiter plate is then incubated with shaking overnight at 37° C.

The recombinant VEGFR-2 protein (1 μg/ml in PBS) is allowed to coat onto the 96 well MaxiSorp™ flat bottom plates (Nunc) overnight at 4° C. As a control, the pure streptavidin is coated onto a separate MaxiSorp™ plate at 2 μg/ml in PBS.

On the following day, liquid in the protein coated MaxiSorp™ plates is discarded, and each well is blocked with 300 ml of 5% skim milk solution at 4° C. for overnight (alternatively, 1 hour at room temperature). The skim milk solution is discarded, and the wells are washed three times with the PBST solution. After the last washing step, about 50 ml of PBS-T containing 4% powdered skim milk is added to each well of the protein coated MaxiSorp™ plates.

About 50 μl of overnight cultures from each well in the 96 well microtiter plate is transferred to the corresponding wells of the VEGFR-2 coated plates as well as the control streptavidin coated plates. The 100 μl mixture in the two kinds of plates is incubated for 1 hour at room temperature. The liquid is discarded from the MaxiSorp™ plates, and the wells are washed about three times with PBST. The HRP-conjugated anti-M13 antibody (Amersham Pharmacia Biotech) is diluted to about 1:7,500, and 100 μl of the diluted solution is added to each well of the MaxiSorp™ plates for 1 hour incubation at room temperature. The liquid is again discarded and the wells are washed about five times with PBS-T. 100 μl of TMB substrate (3, 3′, 5, 5′-tetramethyl-benzidine, Sigma) is added to each well for the color reaction to develop, and the reaction is stopped with 50 μl of the 5N H₂SO₄ solution. The OD₄₅₀ is read on a plate reader (Molecular Devices).

### 5B. DNA Sequencing of the Phage Clones

For each phage clone, the DNA sequencing template is prepared by a PCR method. The following forward/reverse primer pair is used to amplify about 90 nucleotide fragment (Table 9).

TABLE 9

| Primer | DNA base sequence | Remark |
|---|---|---|
| TN6 forward | SEQ ID NO 30: 5′-GGA GGA AGC | R; G or A |
| primer | GGC CGC GGT ACT GGC AGC-3′ | W; A or T |
| | | N; G or A |
| TN6 reverse | SEQ ID NO 31: 5′-CCT CCT CTC | or T |
| primer | TAG AGC GGA CCA GGA GC-3′ | or C |
| | | Y; T or C |
| TN7 forward | SEQ ID NO 32: 5′-GGA GGA AGC | M; A or C |
| primer | GGC CGC ACA GAR GCW GGT-3′ | |
| TN7 reverse | SEQ ID NO 33: 5′-CCT CCT CTC | |
| primer | TAG AGN GGY TCM GTN CC-3′ | |
| TN8 forward | SEQ ID NO 34: 5′-GGA GGA AGC | |
| primer | GGC CGC ACA GAR GCW GGT-3′ | |
| TN8 reverse | SEQ ID NO 35: 5′-CCT CCT CTC | |
| primer | TAG AGN GGY TCM GTN CC-3′ | |
| TN9 forward | SEQ ID NO 36: 5′-GGA GGA AGC | |
| primer | GGC CGC ACA GAR GCW GGT-3′ | |
| TN9 reverse | SEQ ID NO 37: 5′-CCT CCT CTC | |
| primer | TAG AGN GGY TCM GTN CC-3′ | |
| TN10 forward | SEQ ID NO 38: 5′-GGA GGA AGC | |
| primer | GGC CGC GGT ACT GGC AGT-3′ | |
| TN10 reverse | SEQ ID NO 39: 5′-CCT CCT CTC | |
| primer | TAG AGA GGA CCC GGG GC-3′ | |
| TN11 forward | SEQ ID NO 40: 5′-GGA GGA AGC | |
| primer | GGC CGC CGT CCT GGT AGC-3′ | |
| TN11 reverse | SEQ ID NO 41: 5′-CCT CCT CTC | |
| primer | TAG AGA GGA CGA GGA GC-3′ | |
| TN12 forward | SEQ ID NO 42: 5′-GGA GGA AGC | |
| primer | GGC CGC GGC ACC GGT GAC-3′ | |
| TN12 reverse | SEQ ID NO 43: 5′-CCT CCT CTC | |
| primer | TAG AGA GGG CCA GGA TC-3′ | |

The mixture as Table 10 below is prepared for PCR reactions of the individual clones.

TABLE 10

| Reagents | Volume per tube (μl) |
|---|---|
| dH₂O | 36.25 |
| 10X PCR Buffer (w/o MgCl₂) | 5 |
| 25 mM MgCl₂ | 4 |
| 10 mM dNTP mix | 1 |
| 100 μM forward primer | 0.25 |
| 100 μM reverse primer | 0.25 |
| 5U Taq polymerase | 0.25 |
| Phage in TE (section 4) | 3 |
| Final reaction volume | 50 |

The thermocycler (GeneAmp PCR System 9700, Applied Biosystem) is used to run the following program: 94° C. for 5 min; [94° C. for 30 sec, 55° C. for 30 sec, 72° C. for 30 sec]×30 cycles; 72° C. for 7 min; cool to 4° C. The PCR product from each reaction is cleaned up by using the QIAquick Multiwell PCR Purification kit (Qiagen), following the manufacturer's protocol.

The PCR cleaned up product is checked by running 10 μl of each PCR reaction on a 1% agarose gel. The DNA base sequences of the remaining product is then assayed.

### 6. DNA Base Sequence Ranking and Consensus Base Sequence Determination

### 6A. DNA Base Sequence Ranking and Peptide Synthesis

The analyzed DNA base sequences translated from variable nucleotide sequences (section 5B) are correlated to ELISA data of the above 5A section. The clones showing high OD₄₅₀ in the VEGFR-2 coated wells and low OD₄₅₀ in the streptavidin coated wells are considered more important. The sequences occurred multiple times are also considered important. Candidate sequences are chosen based on these criteria and peptides are synthesized in vitro by a known method (Peptron Inc., prepared on commission) for further analysis as bio-active peptides.

### 6B. VEGF:VEGFR-2 Neutralization ELISA (Competitive ELISA)

Competitive ELISA is performed using the peptides synthesized above in Section 6A in order to assess the molecular interaction of the binders. 96-well MaxiSorp™ plates (Nunc) are coated with recombinant human VEGF 165 (R&D Systems) dissolved in DMEM culture medium containing 50 μg/ml BSA. The concentration of the coated VEGF 165 is equivalent to 75% of the maximum binding concentration with 1 nM recombinant VEGFR-2. Then the plate is washed five times with PBS containing 0.1% Tween-20, and blocked at room temperature for two hours with PBS containing 5% BSA. For VEGF:VEGFR-2 neutralization ELISA, each of the peptides synthesized in Section 6A above are diluted to one-fourth each time from 100 nM with PBS containing 1 nM VEGFR-2 and 1% BSA, and adjusted to 6.1 pM. 100 μl of peptide/VEGFR-2 solution is added to each well and the plate is left to react at room temperature for two hours. Subsequently, it is washed five times with PBS containing 0.1% Tween-20, mouse anti-VEGFR-2 antibodies (R&D Systems) is diluted to about 1:3,000 with PBS containing 1% BSA, and left to react at room temperature for one hour. Then 100 μl of goat anti-mouse-IgG-HRP (Pierce) diluted to about 1:10,000 with PBS containing 1% BSA is added to each well of the plate, left to react at room temperature for one hour, and washed five times with PBS containing 0.1% Tween-20. Lastly, 100 μl of TMB substrate (SIGMA) is added to each well of the plate to induce color reaction. The reaction is stopped by adding 50 μl of 5N H₂SO₄ solution, and the OD₄₅₀ value is read on a plate reader (Molecular Devices). Through the foregoing, the IC₅₀ value for the VEGF:VEGFR-2 binding capacity is obtained. The results are as shown in Table 11 below.

TABLE 11

| | 50% inhibition concentration |
|---|---|
| | (IC₅₀, nM) for VEGF:VEGFR-2 |
| Name of Peptide | binding capacity |
| Unrelated control group | >100 |
| DTN6VEGFR-2-1 | 56.747 |
| DTN8VEGFR-2-2 | 49.523 |
| DTN9VEGFR-2-3 | 61.889 |
| DTN10VEGFR-2-4 | 46.889 |
| DTN11VEGFR-2-5 | 50.652 |

### 7. Cell-Based Functional Assay

The peptides synthesized in Section 6A above are screened by cell-based functional assay for inhibition activity. The method described in Example 1, Section 7 is used, except that 5 μM synthesized peptides are added instead of anti-Ang-2 antibodies. The results are as shown in Table 12 below.

TABLE 12

| | Length of the entire tube of HUVEC |
|---|---|
| Name of Peptide | (2 × 10⁴ cells) (pixels) |
| Unrelated control group | 12,227 ± 1,232 |
| DTN6VEGFR-2-1 | 11,093 ± 1,308 |
| DTN8VEGFR-2-2 | 10,891 ± 1,014 |
| DTN9VEGFR-2-3 | 12,101 ± 869 |
| DTN10VEGFR-2-4 | 10,773 ± 992 |
| DTN11VEGFR-2-5 | 11,896 ± 1,159 |

### Example 3

### Preparation of Fused Protein Synergibody

### 1. Preparation of Fused Protein Synergibodies

The four types of chimeric Anti-Ang-2 antibody genes finally selected in Example 1 above are fused with the five types of peptide genes selected in Example 2 above by using recombinant DNA technology, and expression vectors for twenty types of fused protein synergy bodies were prepared. In this example, the peptides are bound to the C-terminal of the heavy chains and the results are as shown in Table 13 below. They are expressed and purified using the method described in Example 1, Section 6. In particular, the heavy chain variable region gene of SEQ ID NOs: 44, 46, 48 or 50 is cloned into a vector including Fc region and constant region of human IgG1. Then, the C-terminal of the Fc region of the vector is cleaved with restriction enzymes NotI (Roche) and XbaI (Roche), and the bioactive peptide gene of Table 3 is inserted and ligated using T4 DNA Ligase (New England Biolab). The light chain variable region gene of SEQ ID NOs: 45, 47, 49 or 51 is cloned into another vector including constant region of human IgG1. The heavy chain-bioactive peptide vector, and the light chain vector obtained in accordance with the above are transfected into HEK-293E cells (Korea Research Institute of Bioscience and Biotechnology).

TABLE 13

| Name of fused protein synergibody | Antibody-peptide fusion type |
|---|---|
| Syn-Ang-VEGFR-1 | SAITAng-2-1 × DTN6VEGFR-2-1 |
| Syn-Ang-VEGFR-2 | SAITAng-2-1 × DTN8VEGFR-2-2 |
| Syn-Ang-VEGFR-3 | SAITAng-2-1 × DTN9VEGFR-2-3 |
| Syn-Ang-VEGFR-4 | SAITAng-2-1 × DTN10VEGFR-2-4 |
| Syn-Ang-VEGFR-5 | SAITAng-2-1 × DTN11VEGFR-2-5 |
| Syn-Ang-VEGFR-6 | SAITAng-2-2 × DTN6VEGFR-2-1 |
| Syn-Ang-VEGFR-7 | SAITAng-2-2 × DTN8VEGFR-2-2 |
| Syn-Ang-VEGFR-8 | SAITAng-2-2 × DTN9VEGFR-2-3 |
| Syn-Ang-VEGFR-9 | SAITAng-2-2 × DTN10VEGFR-2-4 |
| Syn-Ang-VEGFR-10 | SAITAng-2-2 × DTN11VEGFR-2-5 |
| Syn-Ang-VEGFR-11 | SAITAng-2-3 × DTN6VEGFR-2-1 |
| Syn-Ang-VEGFR-12 | SAITAng-2-3 × DTN8VEGFR-2-2 |
| Syn-Ang-VEGFR-13 | SAITAng-2-3 × DTN9VEGFR-2-3 |
| Syn-Ang-VEGFR-14 | SAITAng-2-3 × DTN10VEGFR-2-4 |
| Syn-Ang-VEGFR-15 | SAITAng-2-3 × DTN11VEGFR-2-5 |
| Syn-Ang-VEGFR-16 | SAITAng-2-4 × DTN6VEGFR-2-1 |
| Syn-Ang-VEGFR-17 | SAITAng-2-4 × DTN8VEGFR-2-2 |
| Syn-Ang-VEGFR-18 | SAITAng-2-4 × DTN9VEGFR-2-3 |
| Syn-Ang-VEGFR-19 | SAITAng-2-4 × DTN10VEGFR-2-4 |
| Syn-Ang-VEGFR-20 | SAITAng-2-4 × DTN11VEGFR-2-5 |

### 2. Testing the Activity of Synergibodies

### 2A. Ang-2:Tie-2 Neutralization ELISA (Competitive ELISA)

The method described in Example 1, Section 3 above is used, except that, in the present example, each fused protein synergibodies are diluted from 100 nM to one-fourth each time with PBS containing 1% BSA and 1 nM hAng-2 and adjusted to 6.1 pM for Ang-2:Tie-2 neutralization ELISA. The results are as shown in Table 14 below.

TABLE 14

| | 50% inhibition concentration |
|---|---|
| Name of fused protein | (IC₅₀, nM) for Ang-2:Tie-2 |
| synergibody | binding capacity |
| Unrelated control group | >100 |
| Syn-Ang-VEGFR-1 | 0.773 |
| Syn-Ang-VEGFR-2 | 0.801 |
| Syn-Ang-VEGFR-3 | 0.783 |
| Syn-Ang-VEGFR-4 | 0.812 |
| Syn-Ang-VEGFR-5 | 0.799 |
| Syn-Ang-VEGFR-6 | 1.913 |
| Syn-Ang-VEGFR-7 | 2.012 |
| Syn-Ang-VEGFR-8 | 2.223 |
| Syn-Ang-VEGFR-9 | 1.946 |
| Syn-Ang-VEGFR-10 | 1.997 |
| Syn-Ang-VEGFR-11 | 0.858 |
| Syn-Ang-VEGFR-12 | 0.833 |
| Syn-Ang-VEGFR-13 | 0.903 |
| Syn-Ang-VEGFR-14 | 0.887 |
| Syn-Ang-VEGFR-15 | 0.925 |
| Syn-Ang-VEGFR-16 | 1.216 |
| Syn-Ang-VEGFR-17 | 0.998 |
| Syn-Ang-VEGFR-18 | 1.389 |
| Syn-Ang-VEGFR-19 | 1.276 |
| Syn-Ang-VEGFR-20 | 1.348 |

### 2B. VEGF:VEGFR-2 Neutralization ELISA (Competitive ELISA)

The method in Example 2, Section 6B above is used, except that, in the present example, each fused protein synergibodies are diluted from 100 nM to one-fourth each time with PBS containing 1% BSA and 1nM VEGFR-2 and adjusted to 6.1 pM for VEGF:VEGF-2 neutralization ELISA. The results are as shown in Table 15 below.

TABLE 15

| | 50% inhibition activity |
|---|---|
| Name of Fused Protein | (IC₅₀, nM) on VEGF:VEGFR-2 |
| Synergibody | binding capacity |
| Unrelated control group | >100 |
| Syn-Ang-VEGFR-1 | 3.367 |
| Syn-Ang-VEGFR-2 | 2.968 |
| Syn-Ang-VEGFR-3 | 3.174 |
| Syn-Ang-VEGFR-4 | 2.848 |
| Syn-Ang-VEGFR-5 | 3.084 |
| Syn-Ang-VEGFR-6 | 1.559 |
| Syn-Ang-VEGFR-7 | 1.763 |
| Syn-Ang-VEGFR-8 | 1.476 |
| Syn-Ang-VEGFR-9 | 2.045 |
| Syn-Ang-VEGFR-10 | 2.159 |
| Syn-Ang-VEGFR-11 | 0.872 |
| Syn-Ang-VEGFR-12 | 0.854 |
| Syn-Ang-VEGFR-13 | 0.914 |
| Syn-Ang-VEGFR-14 | 0.889 |
| Syn-Ang-VEGFR-15 | 1.047 |
| Syn-Ang-VEGFR-16 | 1.162 |
| Syn-Ang-VEGFR-17 | 1.048 |
| Syn-Ang-VEGFR-18 | 1.648 |
| Syn-Ang-VEGFR-19 | 1.312 |
| Syn-Ang-VEGFR-20 | 1.380 |

### 3. Cell-Based Functional Assay

The fused protein synergibodies prepared in Section 1 above are screened using cell-based functional assay for inhibition activity. The method described in Example 1, Section 7 is used, except that, in this example, 5 μM of fused protein synergibodies are added instead of anti-Ang-2 antibodies. The results are as shown in Table 16.

TABLE 16

| Name of fused protein | Length of entire tube of HUVEC |
|---|---|
| synergibody | (2 × 10⁴ cells) (pixels) |
| Unrelated control group | 12,227 ± 1,232 |
| Syn-Ang-VEGFR-1 | 4,345 ± 952 |
| Syn-Ang-VEGFR-2 | 3,995 ± 896 |
| Syn-Ang-VEGFR-3 | 5,043 ± 1,183 |
| Syn-Ang-VEGFR-4 | 4,225 ± 1,035 |
| Syn-Ang-VEGFR-5 | 4,065 ± 927 |
| Syn-Ang-VEGFR-6 | 6,157 ± 1,141 |
| Syn-Ang-VEGFR-7 | 5,935 ± 995 |
| Syn-Ang-VEGFR-8 | 6,244 ± 935 |
| Syn-Ang-VEGFR-9 | 6,055 ± 779 |
| Syn-Ang-VEGFR-10 | 5,898 ± 1,037 |
| Syn-Ang-VEGFR-11 | 3,925 ± 675 |
| Syn-Ang-VEGFR-12 | 3,899 ± 923 |
| Syn-Ang-VEGFR-13 | 4,216 ± 747 |
| Syn-Ang-VEGFR-14 | 4,183 ± 698 |
| Syn-Ang-VEGFR-15 | 4,116 ± 815 |
| Syn-Ang-VEGFR-16 | 5,022 ± 809 |
| Syn-Ang-VEGFR-17 | 5,215 ± 714 |
| Syn-Ang-VEGFR-18 | 5,723 ± 736 |
| Syn-Ang-VEGFR-19 | 5,329 ± 822 |
| Syn-Ang-VEGFR-20 | 5,512 ± 699 |

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. While the present invention has been particularly shown and described with reference to exemplary embodiments thereof, it will be understood by those of ordinary skill in the art that various changes in form and details may be made therein without departing from the spirit and scope of the present invention as defined by the following claims.

1. An antibody-peptide fused synergibody comprising:
an antibody; and
a bioactive peptide,
wherein the antibody and the bioactive peptide function in the occurrence or the progress of the identical disease.

2. The antibody-peptide fused synergibody according to claim 1, wherein the antibody and the bioactive peptide effect on different targets.

3. The antibody-peptide fused synergibody according to claim 1, wherein the antibody and the bioactive peptide function in the progress of angiogenesis.

4. The antibody-peptide fused synergibody according to claim 1, wherein the disease is cancer.

5. The antibody-peptide fused synergibody according to claim 1, wherein the antibody is an antibody that has a binding affinity to Ang-2 or a fragment thereof, and the bioactive peptide is a peptide that has a binding affinity to VEGF.

6. The antibody-peptide fused synergibody according to claim 5, wherein the bioactive peptide comprises about 7 to about 50 amino acids.

7. The antibody-peptide fused synergibody according to claim 5, wherein the bioactive peptide is linked to the C-terminus of the heavy chain of the antibody.

8. The antibody-peptide fused synergibody according to claim 1, wherein the antibody comprises
a heavy chain comprising CDR1 having any one or more sequence selected from the group consisting of SEQ ID NO: 1, 4, 7 and 10; CDR2 having any one or more sequence selected from the group consisting of SEQ ID NO: 2, 5, 8 and 11; CDR3 having any one or more sequence selected from the group consisting of SEQ ID NO 3, 6, 9 and 12; and
a light chain comprising CDR1 having any one or more sequence selected from the group consisting of SEQ ID NO: 13, 16, 19 and 22; CDR2 having any one or more sequence selected from the group consisting of SEQ ID NO: 14, 17, 20 and 23; CDR3 having any one or more sequence selected from the group consisting of SEQ ID NO: 15, 18, 21 and 24.

9. The antibody-peptide fused synergibody according to claim 8, wherein the antibody is one or more selected from the group consisting of
an antibody comprising a heavy chain comprising CDR1 of SEQ ID NO: 1, CDR2 of SEQ ID NO: 2 and CDR3 of SEQ ID NO: 3, and a light chain comprising CDR1 of SEQ ID NO: 13, CDR2 of SEQ ID NO: 14 and CDR3 of SEQ ID NO: 15;
an antibody comprising a heavy chain comprising CDR1 of SEQ ID NO: 4, CDR2 of SEQ ID NO: 5 and CDR3 of SEQ ID NO: 6, and a light chain comprising CDR1 of SEQ ID NO: 16, CDR2 of SEQ ID NO: 17 and CDR3 of SEQ ID NO: 18;
an antibody comprising a heavy chain comprising CDR1 of SEQ ID NO: 7, CDR2 of SEQ ID NO: 8 and CDR3 of SEQ ID NO: 9, and a light chain comprising CDR1 of SEQ ID NO: 19, CDR2 of SEQ ID NO: 20 and CDR3 of SEQ ID NO: 21; and
an antibody comprising a heavy chain comprising CDR1 of SEQ ID NO: 10, CDR2 of SEQ ID NO: 11 and CDR3 of SEQ ID NO: 12, and a light chain comprising CDR1 of SEQ ID NO: 22, CDR2 of SEQ ID NO: 23 and CDR3 of SEQ ID NO: 24.

10. The antibody-peptide fused synergibody according to claim 1, wherein the antibody comprises
a heavy chain comprising a variable region having any one or more sequence selected from the group consisting of SEQ ID NO: 44, 46, 48 and 50; and
a light chain comprising a variable region having any one or more sequence selected from the group consisting of SEQ ID NO: 45, 47, 49 and 51.

11. The antibody-peptide fused synergibody according to claim 1, wherein the peptide is a peptide having any one or more sequence selected from the group consisting of SEQ ID NO: 25, 26, 27, 28 and 29.

12. The antibody-peptide fused synergibody according to claim 1, wherein the antibody is one or more selected from the group consisting of
an antibody comprising a heavy chain comprising CDR1 of SEQ ID NO: 1, CDR2 of SEQ ID NO: 2 and CDR3 of SEQ ID NO: 3, and a light chain comprising CDR1 of SEQ ID NO: 13, CDR2 of SEQ ID NO: 14 and CDR3 of SEQ ID NO: 15;
an antibody comprising a heavy chain comprising CDR1 of SEQ ID NO: 4, CDR2 of SEQ ID NO: 5 and CDR3 of SEQ ID NO: 6, and a light chain comprising CDR1 of SEQ ID NO: 16, CDR2 of SEQ ID NO: 17 and CDR3 of SEQ ID NO: 18;
an antibody comprising a heavy chain comprising CDR1 of SEQ ID NO: 7, CDR2 of SEQ ID NO: 8 and CDR3 of SEQ ID NO: 9, and a light chain comprising CDR1 of SEQ ID NO: 19, CDR2 of SEQ ID NO: 20 and CDR3 of SEQ ID NO: 21; and
an antibody comprising a heavy chain comprising CDR1 of SEQ ID NO: 10, CDR2 of SEQ ID NO: 11 and CDR3 of SEQ ID NO: 12, and a light chain comprising CDR1 of SEQ ID NO: 22, CDR2 of SEQ ID NO: 23 and CDR3 of SEQ ID NO: 24,
the peptide is a peptide having any one sequence selected from the group consisting of SEQ ID NO: 25, 26, 27, 28 and 29.

13. A pharmaceutical composition containing the antibody-peptide fused synergibody according to any one of claims 1 through 12 as an effective ingredient.

14. The pharmaceutical composition according to claim 13, inhibiting the function of a target.

15. The pharmaceutical composition according to claim 13, preventing or treating cancer.

16. The method of preparing antibody-peptide fused synergibody according to any one of claims 1 through 12, comprising:
inserting a nucleic acid molecule encoding the antibody-peptide fused synergibody according to any one of claims 1 through 12 into a vector;
introducing the vector into host cells; and
culturing the host cells.

17. The method of preparing antibody-peptide fused synergibody according to claim 16, wherein the inserting a nucleic acid molecule comprises screening an antibody that functions in the occurrence and progress of a target disease; screening a bioactive peptide that functions in the occurrence and progress of the identical disease; obtaining a nucleic acid molecule combining the gene that encodes the screened antibody and the gene that encodes the screened bioactive peptide; and inserting the nucleic acid molecule into the vector.

18. The method of preparing antibody-peptide fused synergibody according to claim 16, wherein the inserting a nucleic acid molecule comprises screening an antibody having a binding affinity to Ang-2; screening a bioactive peptide that has a binding affinity to VEGFR-2; obtaining a nucleic acid molecule combining the gene that encodes the screened antibody and the gene that encodes the screened peptide; and inserting the nucleic acid molecule into the vector.

19. The method of preparing antibody-peptide fused synergibody according to claim 16, wherein the bioactive peptide is screened from a phage display library.

20. A CDR domain of a heavy chain of an antibody having a binding affinity to Ang-2 or a fragment thereof, comprising one or more selected from the group consisting of the amino acid sequences of SEQ ID NO: 1, 2, 3, 4, 5, 6, 7, 8, 9, 10, 11 and 12.

21. A CDR domain of a light chain of an antibody having a binding affinity to Ang-2 or a fragment thereof, comprising one or more selected from the group consisting of the amino acid sequences of SEQ ID NO: 13, 14, 15, 16, 17, 18, 19, 20, 21, 22, 23 and 24.

22. A variable region of a heavy chain of an antibody having a binding affinity to Ang-2 or a fragment thereof, which comprises one or more selected from the group consisting of amino acid sequences of SEQ ID NO: 44, 46, 48 and 50.

23. A variable region of a light chain of an antibody having a binding affinity to Ang-2 or a fragment thereof, which comprises one or more selected from the group consisting of amino acid sequences of the of SEQ ID NO: 45, 47, 49 and 51.

24. A polypeptide having a binding affinity to VEGF, which comprises one or more selected from the group consisting of amino acid sequences of SEQ ID NO: 25, 26, 27, 28 and 29.

25. A recombinant vector comprising
the CDR of claims 20 or 21;
the variable region of claims 22 or 23; or
the polypeptide of claim 24.

26. A transformed host cell comprising the recombinant vector of claim 25.

27. An isolated nucleic acid molecule encoding
the CDR of claims 20 or 21;
the variable region of claims 22 or 23; or
the polypeptide of claim 24.
